(19) United States
(12) Patent Application Publication
(10) Pub. No.: US 2006/0085074 A1
(43) Pub. Date: Apr. 20, 2006

(54) Medical device systems for the spine

(76) Inventor: Kamshad Raiszadeh, Rancho Santa Fe, CA (US)

Correspondence Address: FISH & RICHARDSON PC, P.O. BOX 1022, MINNEAPOLIS, MN, 55440-1022, US

(21) Appl. No.: 11/231,333

(22) Filed: Sep. 19, 2005

Related U.S. Application Data
(63) Continuation-in-part of application No. 10/967,417, filed on Oct. 18, 2004.

(51) Int. Cl. A61F 2/44

(52) U.S. Cl. 623/17.12; 623/17.16; 606/61; 606/90

(57) ABSTRACT

Medical device systems for treating a spine and related methods are described. In some embodiments, a medical device system includes an expandable intradiscal portion configured to be placed between two vertebras, and an expandable first extradiscal portion capable of being in fluid communication with the intradiscal portion.

### CROSS-REFERENCE TO RELATED APPLICATIONS

This application is a continuation-in-part application of and claims priority to U.S. application Ser. No. 10/967,417, filed on Oct. 18, 2004, and entitled “Medical Device Systems for the Spine”, which is hereby incorporated by reference.

### TECHNICAL FIELD

The invention relates to medical device systems for the spine, and related methods.

### BACKGROUND

The human spine includes a series of vertebras. Adjacent vertebras are separated by an anterior intervertebral disc and two posterior facets joints. Together, the disc and facet joints create a spinal motion segment that allows the spine to flex, rotate, and bend laterally. The intervertebral disc also functions as a spacer and a shock absorber. As a spacer, the disc provides proper spacing that facilitates the biomechanics of spinal motion and prevents compression of spinal nerves. As a shock absorber, the disc allows the spine to compress and rebound during activities, such as jumping and running, and resists the axial pressure of gravity during prolonged sitting and standing.

Sometimes, the disc and facets can degenerate, for example, due to the natural process of aging, and produce large amounts of pain. A number of procedures have been developed to treat degeneration of the spinal motion segment. For example, the disc can be removed by discectomy procedure, the disc can be replaced by disc arthroplasty, or the vertebras directly adjacent to the disc can be fused together.

### SUMMARY

In one aspect, described herein are medical device systems for treating a spine, in particular the spinal motion segment, i.e., disc and facets. When implanted in the body, the systems can (i) recreate the biomechanics and kinematics of a functional spinal segment and/or (ii) act as a shock absorber. As a result, the systems allow the spine to move naturally, for example, flex, rotate, and bend laterally. Furthermore, as discussed below, the medical device systems are also capable of treating or reducing pain caused by certain interactions of vertebras.

In another aspect, described herein are methods of implanting medical device systems for treating a spine. In some embodiments, the systems can be implanted using posterior approach techniques and/or through minimally invasive techniques. As a result, recovery time can be reduced and/or the occurrence of pain can be reduced. The medical device systems can also be adjusted (e.g., fine tuned post-operatively) to meet the patient's needs. For example, in certain embodiments, medical device systems disclosed herein include a valve that allows fluid levels within the medical device system to be adjusted post-operatively.

In another aspect, the invention features a medical device system, including an expandable intradiscal portion configured to be placed between two vertebras, and an expandable first extradiscal portion capable of being in fluid communication with the intradiscal portion.

In another aspect, the invention features a medical device system, including an expandable intradiscal portion configured to be placed between two vertebras, an expandable first extradiscal portion capable of being in fluid communication with the intradiscal portion, and an expandable second extradiscal portion in fluid communication with the intradiscal portion and the first extradiscal portion.

In another aspect, the invention features a medical device system, including a flexible first member having an expandable intradiscal portion configured to be placed between two vertebras, and an expandable first extradiscal portion capable of being in fluid communication with the intradiscal portion; and a constraint configured to receive a portion of the first member, the constraint capable of preventing the portion of the first member from extending.

In another aspect, the invention features a medical device system, including an expandable intradiscal portion configured to be placed between two vertebras and to contact one or more of the two vertebra, and a valve capable of being in fluid communication with the intradiscal portion, wherein the valve allows for fluid in the medical device system to be adjusted post-operatively when the medical device system is implanted in the body.

In another aspect, the invention features a method, including providing a medical device system having a first expandable portion and a second expandable portion capable of being in fluid communication with the first expandable portion; positioning the first expandable portion between two vertebras; and positioning the second expandable portion spaced from the vertebras. The second expandable portion can be positioned, for example, in between the spinous processes or directly between the facet joints.

In another aspect, the invention features a method, including removing at least a portion of a disc in a disc space between two vertebras; using a posterior approach to position a first expandable portion of a medical device system in the disc space between the two vertebras; and using a posterior approach to position a second expandable portion of the medical device system posterior to the disc space.

Other aspects, features and advantages of the invention will be apparent from the description of the embodiments thereof and from the claims.

### BRIEF DESCRIPTION OF DRAWINGS

FIG. 1 is a schematic view of a portion of an embodiment of a medical device system between two vertebras.

FIG. 2A is a schematic lateral view of the medical device system of FIG. 1 attached to the two vertebras; and FIG. 2B is a schematic posterior view of the medical device system of FIG. 1 attached to the vertebras.

FIGS. 3A, 3B, 3C, and 3D illustrate an embodiment of a method of implanting the medical device system of FIG. 1.

FIG. 4 is a partial schematic view of a portion of an embodiment of a medical device system.

FIG. 5 is a partial schematic view of a portion of an embodiment of a medical device system.

FIG. 6 is a partial schematic view of a portion of an embodiment of a medical device system.

FIG. 7 is a partial schematic view of a portion of an embodiment of a medical device system.

FIG. 8 is a partial schematic view of a portion of an embodiment of a medical device system.

FIG. 9A is a schematic lateral view of an embodiment of a medical device system; and FIG. 9B is a schematic coronal view of the medical device system of FIG. 9A.

FIG. 10 is a schematic coronal view of an embodiment of a medical device system.

FIG. 11 is a schematic coronal view of an embodiment of a medical device system.

### DETAILED DESCRIPTION

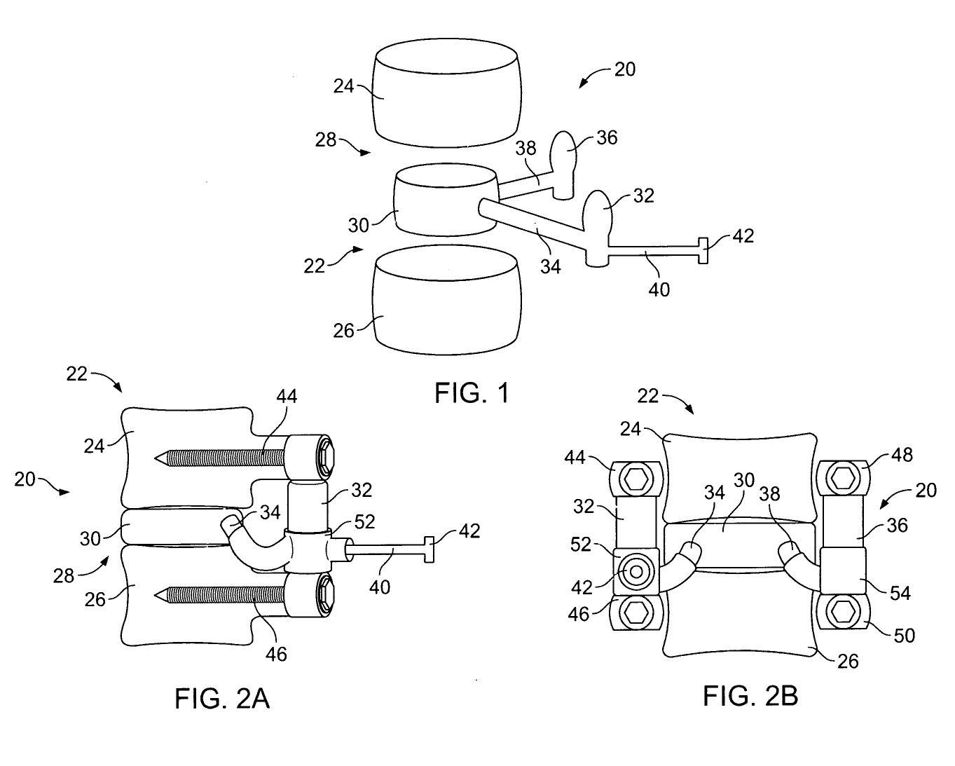

Referring to FIGS. 1, 2A, and 2B, a medical device system 20 is shown along a spinal segment 22 between a superior vertebra 24 and an inferior vertebra 26. Medical device system 20 includes an elongated member 28 having an expandable intradiscal portion 30, a first expandable extradiscal portion 32 in fluid communication with the intradiscal portion via a first hollow conduit 34, and a second extradiscal portion 36 in fluid communication with the intradiscal portion via a second hollow conduit 38. Elongated member 28 further includes a hollow filler tube 40 and a valve 42 for filling the elongated member with a fluid, such as saline, to a predetermined pressure. System 20 further includes multiple (as shown in FIGS. 2A and 2B, four) pedicle screws 44, 46, 48, and 50 that attach the system to the spinal segment 22, and one or more (as shown, two) constraints 52 and 54 that surround portions of elongated portion 28 to prevent the portion(s) from expanding. As shown, elongated member 28 is secured to spinal segment 22 with intradiscal portion 30 positioned between vertebras 24 and 26 (for example, in place of a portion of an intervertebral disc), and extradiscal portions 32 and 36 positioned away from (as shown, posterior of) the intravertebral disc.

In use, medical device system 20 is capable of mimicking an intervertebral disc to allow spinal segment 22 to move normally. In particular, system 20 uses the hydraulic pressure from the fluid filled in elongated member 28 to stabilize spinal segment 22 during motion. For example, when the patient bends or flexes forward, this movement can compress intradiscal portion 30, thereby transferring fluid by hydraulic pressure from the intradiscal portion to one or both of extradiscal portions 32 and 36 via conduits 34 and/or 38. One or both of extradiscal portions 32 and 36 can expand as a result of the additional fluid. The expansion of extradiscal portions 32 and 36 can increase the forces of distraction of the vertebras or decrease the forces of distraction, for example, by controlling the manner in which the extradiscal portion(s) deform. When the patient bends or flexes backward, this movement can compress one or both of extradiscal portions 32 and/or 36, thereby transferring fluid by hydraulic pressure from the extradiscal portion(s) to intradiscal portion 30, which can expand as a result of the additional fluid. Similarly, when the patient rotates or bends laterally, fluid from one of extradiscal portions 32 or 36 can flow to and expand intradiscal portion 30 and/or the other extradiscal portion. Thus, medical device system 20 is capable of allowing spinal segment 22, such as a lumbar spinal segment, to move, for example, flex, rotate, and/or bend, relatively naturally while still maintaining mechanical integrity and stability.

What is more, intradiscal portion 30 can act as a spacer and a shock absorber between vertebras 24 and 26. For example, intradiscal portion 30 can prevent spinal nerves from pinching, and/or can resiliently cushion compressive forces along the length of the spine. Furthermore, by expanding the intradiscal portion, the vertebral bodies are distracted, resulting in decompression of previously compressed nerves. Compressive forces can occur during activities such as running or jumping, or during prolonged periods of sitting or standing.

As indicated above, elongated member 28 includes intradiscal portion 30 and extradiscal portions 32 and 36. Intradiscal portion 30 is generally configured to be placed, wholly or partially, between two vertebras. In some embodiments, as described below, intradiscal portion 30 can be configured to occupy an intradiscal space, or the volume previously occupied by an intervertebral disc, between the vertebras. Intradiscal portion 30 can wholly or partially occupy the intradiscal space (e.g., just the nucleus of the intradiscal space). In comparison, extradiscal portions 32 and 36 are generally configured not to be placed between two vertebras; rather they are configured to be placed adjacent to the posterior facet joints. Extradiscal portions 32 and 36 can have various configurations, e.g., generally cylindrical, or generally oval. Intradiscal portion 30 and extradiscal portions 32 and 36 are all capable of expanding or compressing as a function of external compression forces and internal fluid pressure.

Elongated member 28 can include (e.g., be formed of) a biocompatible flexible material that can be expanded by internal fluid pressure in the member. The flexibility of the material can allow spinal segment 22 to move relatively naturally. Biocompatible materials used in elongated member 28 are also capable of withstanding stresses applied to an intervertebral disc (e.g., stress forces of 200 pound force/square inch (psi) during lifting and 40-70 psi during normal activities.) In some embodiments, the material can be implanted in the body for an extended period of time, e.g., for several years. In certain embodiments, the elongated member is implanted permanently, and need not be removed.

Examples of flexible biocompatible materials that can be used to form an elongated member 28 include pure polymers, polymer blends, and copolymers. Examples of polymers include nylon, silicon, latex, and polyurethane. For example, the elongated member can be made from materials similar or identical to the high-performance nylon used in the RX Dilation Balloons from Boston Scientific (Natick, Mass.), wherein the material is reinforced or thickened to withstand the forces described herein. Other flexible biocompatible materials include block co-polymers such as castable thermoplastic polyurethanes, for instance, those available under the trade names CARBOTHANE (Thermedics) ESTANE (Goodrich), PELLETHANE (Dow), TEXIN (Bayer), Roylar (Uniroyal), and ELASTOTHANE (Thiocol), as well as castable linear polyurethane ureas, such as those available under the tradenames CHRONOFLEX AR (Cardiotech), BIONATE (Polymer Technology Group), and BIOMER (Thoratec). Other examples are described, e.g., in M. Szycher, J. Biomater. Appl. “Biostability of polyurethane elastomers: a critical review”, 3(2):297-402 (1988); A. Coury, et al., “Factors and interactions affecting the performance of polyurethane elastomers in medical devices”, J. Biomater. Appl. 3(2):130-179 (1988); and Pavlova M, et al., “Biocompatible and biodegradable polyurethane polymers”, Biomaterials 14(13):1024-1029 (1993), the disclosures of which are incorporated herein by reference. Elongated member 28 can optionally include: (i) multiple layers of the same or different materials, (ii) reinforcing materials, and/or (iii) sections of varied thickness designed to withstand the forces described herein. Methods for shaping and forming flexible biocompatible materials, such as casting, co-extrusion, blow molding, and co-blowing techniques, are described, e.g., in “Casting”, pp. 109-110, in Concise Encyclopedia of Polymer Science and Engineering, Kroschwitz, ed., John Wiley & Sons, Hoboken, N.J. (1990), U.S. Pat. Nos. 5,447,497; 5,587,125; 5,769,817; 5,797,877; and 5,620,649, and International Patent Application No. WO002613A1.

Elongated member 28 can be formed as a unitary structure or as an assembly of multiple parts. For example, one or more expandable portions 30, 32, and/or 36 can include one or more expandable materials, and one or more conduits 34 and/or 38 can include one or more relatively rigid, non-expandable materials. Examples of non-expandable materials include metals (such as stainless steels) and rigid biocompatible polymers (such as polypropylene, polyimides, polyamides, polyesters, and ceramics). Expandable portions 30, 32, and/or 36 can include the same material or different materials to provide different expandability characteristics (e.g., to increase or to decrease distraction), and thus different stabilization and performance characteristics. Additionally or alternatively, performance of expandable portions 30, 32, and/or 36 can be changed by changing physical parameters, such as wall thickness, cross-sectional configuration, inner diameter, and/or outer diameter. The parts can be joined together, for example, by gluing and/or by thermally bonding overlapping end portions of the parts.

In embodiments in which conduits 34 and/or 38 include an expandable material, system 20 includes one or more constraints 52 and/or 54 surrounding the conduit(s), as shown in FIGS. 2A and 2B. Constraints 52 and 54 prevent the surrounded portion(s) of elongated member 28 from expanding, thereby allowing only selected portions of the elongated member (such as intradiscal portion 30 and extradiscal portions 32 and 36) to expand and contract as described above. Constraints 52 and 54 can also limit the movement of conduits 34 and/or 38, for example, to prevent the conduit(s) from contacting the patient's spinal nerves. Constraints 52 and 54 can include a rigid material formed, for example, into an L-shape, to surround or to fit over elongated member 28. Examples of rigid materials include metals or alloys (such as stainless steels) or rigid biocompatible polymers Constraints 52 and 54 can wholly or partially surround the selected portion(s) of elongated member 28. Constraints 52 and 54 can be attached to pedicle screws (e.g. 44, 46, 48, or 50). In some embodiments, conduits 34 and/or 38 connect intradiscal portion 30 to extradiscal portions 32 and/or 36 via non-expandable, flexible tubing.

Medical device system 20 further includes filler tube 40, valve 42 and pedicle screws 44, 46, 48, and 50. The pedicle screws are used to anchor elongated member 28 (and constraints 52 and 54, if present) to vertebras 24 and 26. Examples of pedicle screws are available from DepuySpine (Raynham, Mass.), Synthes (Paoli, Pa.), and Sofamor 30 Danek (Memphis, Tenn.). Valve 42 can be any device capable of being used to selectively open and close filler tube 40, for example, to introduce fluid into elongated member 28 or to adjust the fluid pressure in the elongated member. Examples of valve 42 include infusion ports such as those used for the regular administration of medication (e.g., in chemotherapy) and/or regular blood withdrawal. Exemplary infusion ports include PORT-A-CATH from Pharmacia (Piscataway, N.J.); MEDI-PORT from Cormed (Cormed; Medina, N.Y.); INFUSE-A-PORT from Infusaid (Norwood, Mass.), and BARD PORT from Bard Access Systems (Salt Lake City, Utah). Other examples of valve 42 include the PORT-CATH Systems (e.g. PORT-A-CATH Arterial System) available from Smith's Medical MD, Inc. (St. Paul, Minn.). As shown in FIGS. 1, 2A, and 2B, one filler tube 40 is directly connected to extradiscal portion 32, but in other embodiments, one or more filler tubes can be directly connected to extradiscal portion 32, extradiscal portion 36, and/or intradiscal portion 30, in any combination.

The fluid introduced into elongated member 28 can be any biocompatible fluid. The fluid can include one composition or a mixture of compositions that provide one or more desired properties, such as viscosity or density. In some embodiments, the fluid has a viscosity similar to water (e.g., near 1.). The fluid can be a liquid (e.g., saline) or a gel. Embodiments of medical device system 20 and other embodiments of medical device systems described herein can be implanted in patients in need of treatment for spondylolysis, spondylolisthesis, and degenerative disc disease. The medical device systems can also be implanted in patients suffering internal disc disruption and disc herniation.

In certain embodiments, the method of implanting medical device system 20 can be performed completely by a posterior approach to the spine. For example, an uninflated intradiscal portion 30 can be threaded through the posterior aspect of the spine, e.g. through an arthroscopic cannula, to reach the intradiscal space. Extradiscal portions 32 and/or 36 can also be introduced into the patient from a posterior approach since the portion(s) can be positioned posterior to the spine and intradiscal portion 30. In the event that system 20 needs to be adjusted after implantation, the adjustments can also be performed by a posterior approach to the spine. Thus, implantation by posterior approach has the following advantages: (i) easier access to the spine and (ii) the procedure can be repeated. Furthermore, since elongated member 28 can be introduced in an uninflated or partially inflated state, and subsequently filled with fluid, a medical device system 20 can be implanted using minimally invasive techniques that can reduce pain and/or recovery time for the patient.

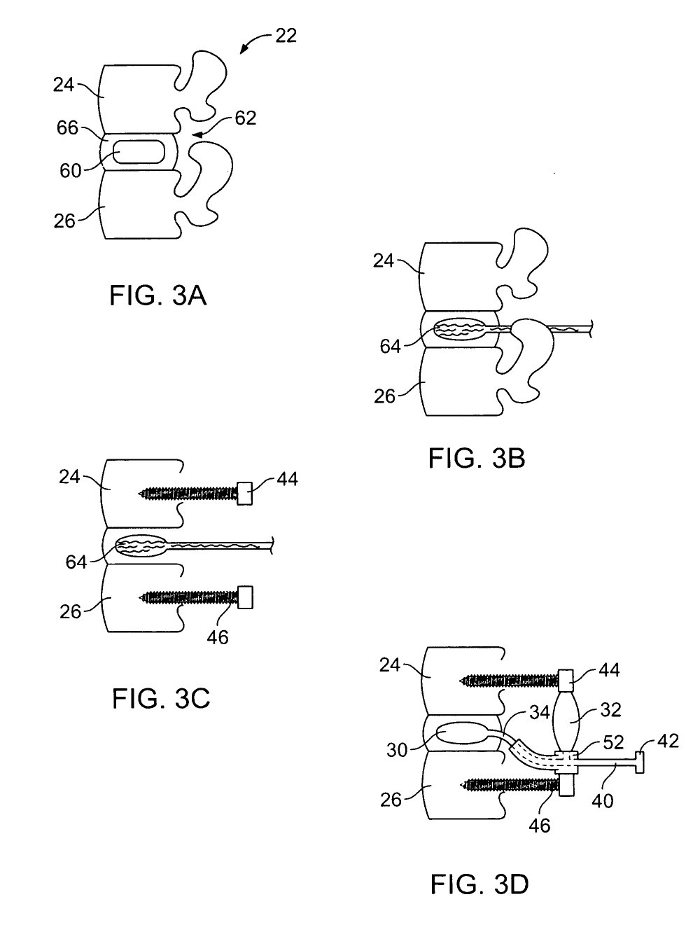

Referring to FIGS. 3A-3D, a method of implanting medical device system 20 is shown. The method in overview includes first forming a disc space 60, e.g., by removing at least a portion of the nucleus of the intervertebral disc 62 (FIG. 3A). Next, disc space 60 is measured. As shown, a test balloon 64 is inserted into disc space 60 to determine the size of the disc space (FIG. 3B). One or more pedicle screws (as shown in FIG. 3C, screws 44 and 46) are then secured to vertebras 24 and 26. Extradiscal portions 32 and 36 can be placed either adjacent to or in place of the facet (i.e., zygapophyseal) joint(s). The remaining components of medical device system 20 are positioned in place and secured to the screws (FIG. 3D).

More specifically, the method includes removing at least a portion of intervertebral disc 62 to prepare the implantation site for medical device system 20. Referring to FIG. 3A, spinal segment 22 includes a disc 62, which includes a nucleus (that has been removed so not shown) surrounded by an annulus 66, located between superior vertebra 24 and inferior vertebra 26. A unilateral or bilateral spinal discectomy can be performed, e.g., with a standard laminectomy or with a minimally invasive lumbar incision posterior to the patient's spine, to remove at least a portion of or as much as possible (e.g., all) of the nucleus to form disc space 60. Generally, enough of the nucleus is removed to allow enough fluid volume inside the balloon to be able to fill extradiscal portions 32 and/or 36. In some embodiments, a portion of or all of annulus 66 is also removed by either a laminectomy or a minimally invasive procedure. Discectomy and laminectomy procedures are described, for example, in Bridwell et al., Eds., “The Textbook of Spinal Surgery, Second Edition,” Lippincott-Raven, Philadelphia, Pa. (1997), which is incorporated herein by reference in its entirety. In some embodiments, when the medical device system is implanted to allow for conversion to a spinal fusion, the cartilaginous end plates in the disc space are curetted and removed.

After disc space 60 is formed, referring to FIG. 3B, the disc space is measured. Test balloon 64 is inserted into disc space 60 to determine the position and volume of the disc space. The position and volume of disc space 60 can be used to determine one or more of the following: (i) that the desired disc space was formed, (ii) the desired disc height to be restored, and (iii) the size and type of intradiscal portion 30 that can be used. Test balloon 64 can be inflated with, for example, (a) a fluid containing a radiopaque marker and detected using X-ray fluoroscopy or (b) a fluid containing a contrast agent (such as an omnipaque-containing material) and detected using intraoperative fluoroscopy.

Next, referring to FIG. 3C, pedicle screws 44, 46, 48, and 50 are secured to vertebras 24 and 26. As shown in FIG. 2B, screws 44 and 48 are secured to the pedicle and vertebral body of superior vertebra 24, and screws 46 and 50 are secured to pedicle and vertebral body of inferior vertebra 26. In some embodiments, a partial or complete facetectomy is performed prior to or after securing screws 46 and 50. Removal of facet joints removes a potential source of pain and facilitates placement of extradiscal portions 32 and 36. Implantation of pedicle screws and facetoctomy procedures are described, for example, in Bridwell et al. 1997, supra.

After pedicle screws 44, 46, 48, and 50 are secured to vertebras 24 and 26, the remaining components of medical device system 20 are connected to the screws. Test balloon 64 is withdrawn from disc space 60, and intradiscal portion 30 is placed into the disc space. Elongated member 28 can be secured to pedicle screws 44, 46, 48, and 50, for example, using biocompatible bonding agents. Referring to FIG. 3D, in embodiments in which system 20 includes constraint(s) 52 and/or 54, portions of elongated member 28, e.g., extradiscal portions 34 and/or 38, can be threaded through the constraint(s), e.g., prior to implanting the system. Constraint(s) 52 and/or 54 can be attached to pedicle screw(s) 46 and/or 50 using biocompatible bonding agents or fastening means. As shown, upon implantation of system 20, intradiscal portion 30 is positioned between vertebras 24 and 26, and extradiscal portions 32 and 36 are positioned posterior of the vertebras. Filler tube 40 and valve 42 are posterior to extradiscal portions 32 and 36.

Fluid is then introduced into elongated member 28 via valve 42 and filler tube 40. The amount of fluid introduced into elongated member 28 can be a function of disc height, and fluid pressure. In some embodiments, fluid is introduced until normal disc height is restored, normal motion is restored, and/or pain is decreased. When the desired amount of fluid has been introduced into elongated member 28, valve 42 is closed to seal the elongated member. In some embodiments, elongated member 28 is partially inflated, e.g., by containing a predetermined amount of fluid, prior to implantation to ease handling and inserting of system 20.

The patient's incisions can then be closed according to conventional methods. Filler tube 40 and valve 42 are positioned posterior to the patient's spine in the subcutaneous space.

As a result of the posterior position of valve 42, the fluid in system 20 can be adjusted relatively easily after the operation, e.g., to affect the performance of the system, or during the implantation operation. For example, additional fluid can be introduced into and/or fluid can be withdrawn from system 20 through filler tube 40 and valve 42 to tune or to optimize the performance of the system. Introducing additional fluid can increase fluid pressure in intradiscal portion 30, thereby increasing its height and the amount of separation between vertebras 24 and 26. Increasing fluid pressure can also increase the rigidity or lower the flexibility of extradiscal portions 32 and 36. Increased pressure in the system can increase the rigidity of the motion segment, thereby allowing treatment of spondylolisthesis or instability from degenerative disc disease. Withdrawing fluid from system 20 can decrease the separation between vertebras 24 and 26, and/or enhance bending, twisting, and/or flexibility of extradiscal portions 32 and 36.

Alternatively or additionally to changing the amount of fluid in system 20, the properties of the fluid, such as its composition, density, or viscosity, can be adjusted to alter the performance of the system. For example, to change the performance of system 20, the existing fluid in the system can be replaced, wholly or in part, with another fluid. One or more fluids can be introduced into system 20 to react with (e.g., to gel with) the existing fluid to change the properties, such as viscosity and/or density, of the fluid.

Adjustment of the fluid can be performed by gaining access to valve 42, for example, by direct injection into valve 42 when valve 42 is an infusion port or by making a small incision under local sedation. Valve 42 can be used to introduce, withdraw, or replace fluid, and subsequently closed to seal elongated member 28.

While a number of embodiments have been described, the invention is not so limited.

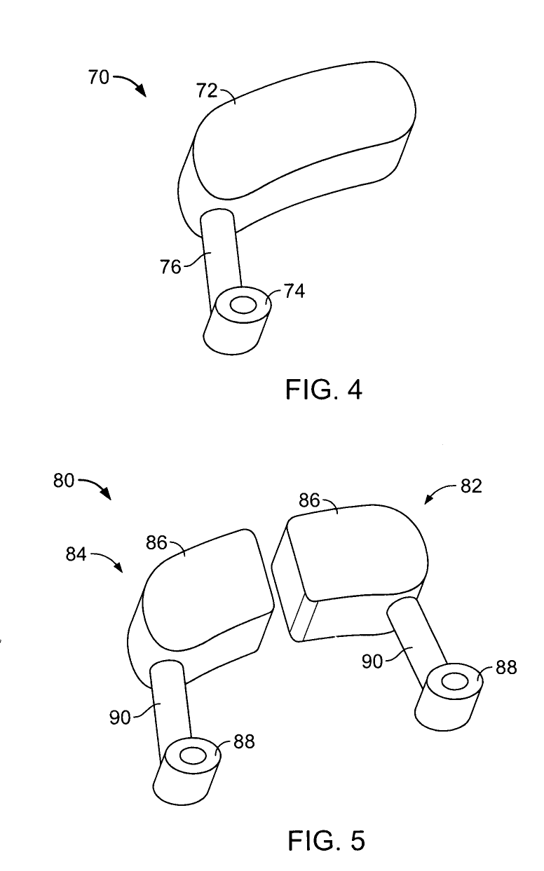

For example, while medical device system 20 is shown above including one expandable intradiscal portion 30 and two expandable extradiscal portions 32 and 36, the medical device system can include other number of expandable portions. Referring to FIG. 4, an elongated member 70 includes one intradiscal portion 72 and one extradiscal portion 74 in fluid communication with the intradiscal portion via a conduit 76. Extradiscal portion 74 can be formed so that it can be implanted on the right side of the spine or on the left side of the spine. Elongated member 70 and its expandable portions 72 and 74 can be generally the same as elongated member 28 and its expandable portions described above. For example, elongated member 70 can include the same material(s) as described above, and conduit 76 can be prevented from expanding using one or more constraints (not shown) as described above. One or more filler tubes and/or one or more valves (not shown) can be directly connected to intradiscal portion 72 and/or extradiscal portion 74. Elongated member 70 can be secured to the spine by attaching extradiscal portion 74 to pedicle screws that are anchored to inferior and superior vertebras using the methods described above. Embodiments of elongated member 70 can be used in patients who have unilateral nerve impingement or when a sufficient amount of the motion segment can be removed and replaced by a unilateral procedure.

In some embodiments, two elongated members 70 can be used together in a medical device system. FIG. 5 shows a portion of a medical device system 80 having a first elongated member 82 and a second elongated member 84. Similar to elongated member 70, each of first elongated member 82 and second elongated member 84 includes an intradiscal portion 86 and an extradiscal portion 88 in fluid communication with the intradiscal portion via a conduit 90. The two intradiscal portions 86 are sized and configured to occupy, wholly or partially, the disc space between two vertebras. As shown, the two intradiscal portions 86 are equally sized and configured, but in other embodiments, the portions can be differently sized and configured, for example, to compensate for scoliosis or asymmetric disc collapse.

Embodiments of medical device system 80 can be used in patients suffering from disc space collapse, bilateral radiculopathy, spondylolisthesis or scoliosis.

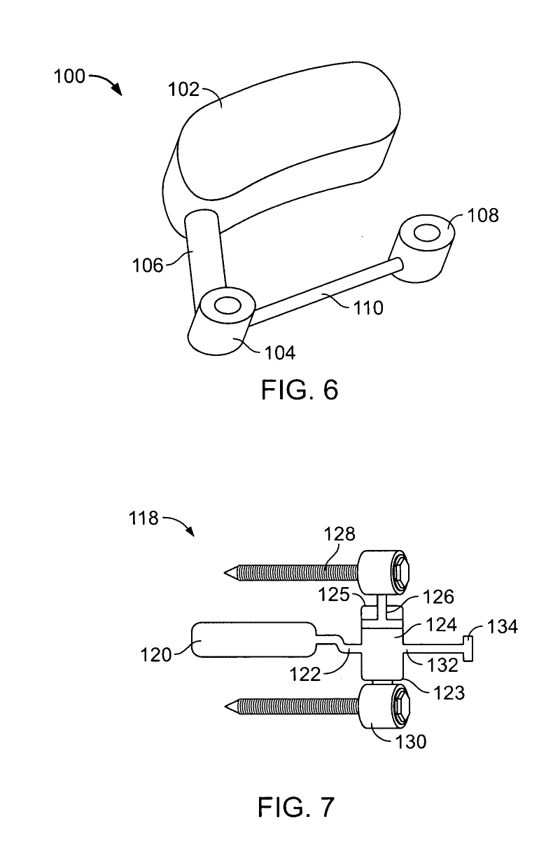

In other embodiments, referring to FIG. 6, an elongated member 100 includes one intradiscal portion 102, a first extradiscal portion 104 in fluid communication with the intradiscal portion 118 via a hollow conduit 106, and a second extradiscal portion 108 in fluid communication with the intradiscal portion through the first intradiscal portion through a second conduit 110. Elongated member 100 and its expandable portions can be generally the same as elongated member 28 and its expandable portions described above. For example, elongated member 100 can include the same material(s) as described above, and conduits 106 and 110 can be prevented from expanding using constraints (not shown) as described above. One or more filler tubes and/or one or more valves (not shown) can be directly connected to intradiscal portion 102 and/or extradiscal portions 104 and 108, in any combination.

Elongated member 100 can be secured to the spine by attaching extradiscal portion 104 and 108 to pedicle screws that are anchored to inferior and superior vertebras using the methods described above. Embodiments of elongated member 100 can be used in patients in which the surgeon deems that unilateral disc removal and replacement is sufficient.

The medical device systems described herein can further include one or more strain or pressure gauges that indicate fluid pressure within the systems. The fluid pressure can be used to determine whether fluid needs to be introduced or withdrawn from the systems, and can indicate whether a system is functioning properly. In some embodiments, a medical device system further includes one or more miniaturized pressure gauges positioned so as to measure fluid pressure within a portion of elongated member 100. Examples of miniaturized pressure gauges include micro-machined devices (i.e., so-called “Micro-Electro-Mechanical Systems” or MEMS) such as piezoresistive pressure sensors and capacitative pressure sensors. An example of a capacitative pressure sensor has been described, for example, in Akar et al., “A Wireless Batch Sealed Absolute Capacitive Pressure Sensor,” Sensors and Actuators Journal 95(1): 29-38 (2001).

In certain patients, the medical device systems described herein can be modified to create a spinal fusion. Spinal fusion is appropriate if treatment with the device should fail, e.g., because of mechanical failure or because the patient's pain continues. Morphogenic products can be placed inside the intradiscal portion 30 and extradiscal portions 32 and/or 36 can be replaced by a rigid rod. Methods of performing a spinal fusion are generally described in Bridwell et al. 1997 supra.

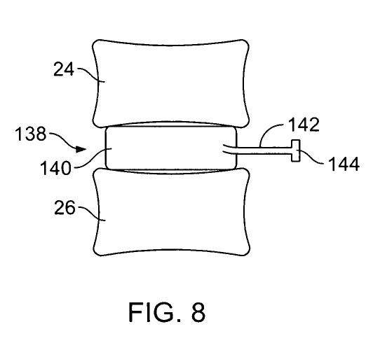

In yet another embodiment, referring to FIG. 8, a medical device system 138 includes an intradiscal portion 140 and a valve 144. As shown, system 138 lacks an extradiscal portion (e.g., element 32 or 36 shown in FIG. 1) between intradiscal portion 140 and valve 144. When implanted between a superior vertebra 24 and an inferior vertebra 26, pressure within intradiscal portion 140 can be adjusted by adding, withdrawing, or changing fluid through valve 144. As depicted in FIG. 8, valve 144 is in fluid communication with intradiscal portion 140 via a hollow filler tube 142. In other embodiments, hollow filler tube 142 is an integrated part of valve 144. In still other embodiments, hollow filler tube 142 can be omitted altogether; and intradiscal portion 140 is linked directly to valve 144. In an additional embodiment, referring to FIG. 7, a medical device system 118 includes an extensible intradiscal portion 120 in fluid communication with an extradiscal portion 124 via a hollow conduit 122. As shown, extradiscal portion 124 includes a piston. Piston arm 126, which extends from a first piston end 125, is attached to an upper pedicle screw 128. A second piston end 123 is attached to a lower pedicle screw 130. Intradiscal portion 120 is configured (i) to wholly or partially occupy a disc space, i.e. a space formerly occupied by a spinal disc, and (ii) to contact the two vertebras (not shown) separated by the disc space. Fluid can be added to, withdrawn from, and/or adjusted in the system through a valve 134 and hollow filler tube 132. In other embodiments, the vertical orientation of piston 124 is reversed and piston arm 126 is attached to lower pedicle screw 130, while the piston end 123, is attached to upper pedicle screw 128. In other embodiments, system 118 includes multiple (e.g., two) extradiscal portions 124 in fluid communication with intradiscal portion 120.

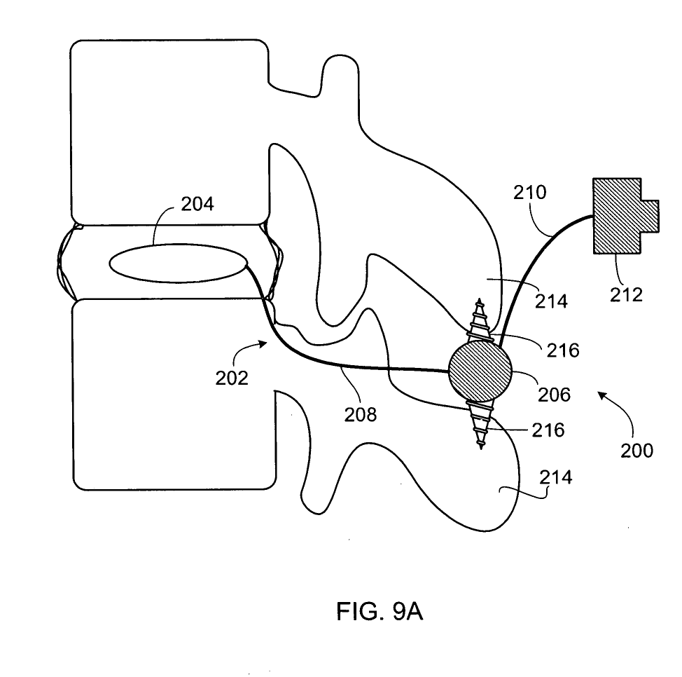

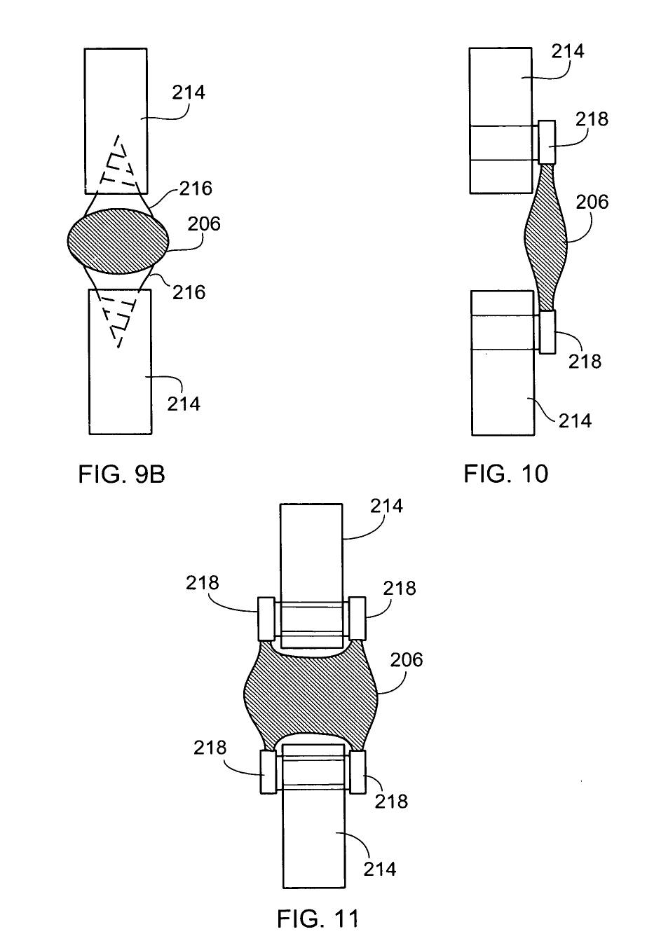

While the extradiscal portion(s) can be secured using one or more pedicle screws, in other embodiments, no pedicle screws are used. Referring to FIGS. 9A and 9B, a medical system 200 includes an elongated member 202 having an expandable intradiscal portion 204, an expandable extradiscal portion 206 in fluid communication with the intradiscal portion 204 via a hollow conduit 208, a hollow filler tube 210 in fluid communication with the extradiscal portion, and a valve 212 for filling and modulating the elongated member 202. As with the other extradiscal portions described herein, expansion of extradiscal portion 206 can increase the forces of distraction of the vertebras or decrease the forces of distraction. Extradiscal portion 206 is secured in the inter-spinous region, as shown, in-line between the spinous processes 214. Extradiscal portion 206 can be secured, for example, by attaching biocompatible (e.g., plastic or metal) connectors 216 to the extradiscal portion (e.g., by an adhesive and/or mechanical bonding), and anchoring the connectors to the vertebras. Connectors 216 can be, for example, screw-like devices or cup-shaped devices that mate with the extradiscal portion. Intradiscal portion 204, extradiscal portion 206, and other components of medical system 200 can be made and modified as generally described above. Other embodiments of medical system 200 are also possible. For example, referring to FIG. 10, extradiscal portion 206 can be between, but not in-line with, the spinous processes 214, as shown, to the side of the spinous processes. Extradiscal portion 206 can be positioned on the left side or on the right side of the spinous processes by using biocompatible anchors 218 that are secured to the spinous processes. In some embodiments, referring to FIG. 11, extradiscal portion 206 can constructed to be placed in-line between the spinous processes 214 and on one or more sides (as shown, both sides) of the spinous processes, as shown, by having a dumb-bell shape. In other embodiments, medical system 200 can have two intradiscal portions (e.g., as shown in FIG. 4) and/or two extradiscal portions (e.g., as shown FIGS. 1, 5, and 6).

All references, such as patents, patent applications, and publications, referred to above are incorporated by reference in their entirety.

Other embodiments are within the scope of the following claims.

What is claimed is:

1. A medical device system, comprising:
an expandable intradiscal portion configured to be placed between two vertebras; and
an expandable first extradiscal portion capable of being in fluid communication with the intradiscal portion.

2. The medical device system of claim 1, wherein the intradiscal portion is configured to be placed in an intradiscal space between two vertebras.

3. The medical device system of claim 1, further comprising a flexible and elongated first member comprising the intradiscal portion and the first extradiscal portion.

4. The medical device system of claim 2, wherein the first member comprises a polymer.

5. The medical device system of claim 2, further comprising a constraint configured to receive a portion of the first member, the constraint capable of preventing the portion of the first member from extending.

6. The medical device system of claim 2, wherein the first member comprises an elongated portion defining a lumen in fluid communication with the intradiscal portion and the first extradiscal portion.

7. The medical device system of claim 2, wherein the first member is adapted to be secured to a screw.

8. The medical device of claim 1, further comprising a valve in fluid communication with intradiscal portion and the first extradiscal portion.

9. The medical device system of claim 1, wherein the intradiscal portion comprises a surface configured to contact at least one of the two vertebras.

10. The medical device system of claim 1, wherein the extradiscal portion is configured to be secured to a spinal process.

11. The medical device system of claim 10, wherein the extradiscal portion is configured to be secured in-line between two spinal processes.

12. A medical device system, comprising:
an expandable intradiscal portion configured to be placed between two vertebras;
an expandable first extradiscal portion capable of being in fluid communication with the intradiscal portion; and
an expandable second extradiscal portion in fluid communication with the intradiscal portion and the first extradiscal portion.

13. The medical device of claim 12, further comprising a flexible and elongated first member comprising the intradiscal portion and the first and second extradiscal portions, the first member comprising a first elongated portion defining a lumen extending between the intradiscal portion and the first extradiscal portion, and a second elongated portion defining a lumen extending between the intradiscal portion and the second extradiscal portion.

14. The medical device system of claim 12, further comprising at least two constraints configured to receive portions of the first member, the constraints capable of preventing the portions of the first member from extending.

15. A medical device system, comprising:
a flexible first member comprising
an expandable intradiscal portion configured to be placed between two vertebras, and
an expandable first extradiscal portion capable of being in fluid communication with the intradiscal portion; and
a constraint configured to receive a portion of the first member, the constraint capable of preventing the portion of the first member from extending.

16. The medical device system of claim 15, wherein the first member further comprises a valve in fluid communication with the intradiscal portion and the extradiscal portion.

17. The medical device system of claim 15, wherein the first member further comprises an expandable second extradiscal portion in fluid communication with the intradiscal portion and the first extradiscal portion.

18. The medical device system of claim 15, wherein the first member comprises a polymer.

19. A medical device system, comprising:
an expandable intradiscal portion configured to be placed between two vertebras and to contact one or more of the two vertebra; and
a valve capable of being in fluid communication with the intradiscal portion, wherein the valve allows for fluid in the medical device system to be adjusted post-operatively when the medical device system is implanted in the body.

20. The medical device system of claim 19, further comprising an extradiscal portion, wherein the extradiscal portion includes a piston, and the extradiscal portion and the intradiscal portion are capable of being in fluid communication with each other.

21. A method, comprising:
providing a medical device system comprising a first expandable portion and a second expandable portion capable of being in fluid communication with the first expandable portion;
positioning the first expandable portion between two vertebras; and
positioning the second expandable portion spaced from the vertebras.

22. The method of claim 21, wherein:
the first expandable portion is positioned in a disc space between two vertebras; and
the second expandable portion is spaced posterior to the disc space.

23. The method of claim 21, wherein the second expandable portion is positioned posterior of the vertebras.

24. The method of claim 21, further comprising securing the second expandable portion to a screw secured to a vertebra.

25. The method of claim 21, wherein the first and second expandable portions are positioned in a body from a posterior approach.

26. The method of claim 21, further comprising removing at least a portion of a disc between the two vertebras.

27. The method of claim 21, further comprising removing at least a portion of a facet joint of at least one of the two vertebras.

28. The method of claim 21, further comprising introducing a fluid into the first expandable portion and the second expandable portion.

29. The method of claim 28, further comprising adjusting the amount of fluid in the medical device system.

30. The method of claim 29, wherein the pressure is adjusted from a posterior approach through a valve in fluid communication with the first and second expandable portions.

31. The method of claim 21, wherein the medical device system comprises a flexible member comprising the first and second expandable portions, and further comprising constraining a portion of the flexible member from extending.

32. The method of claim 21, wherein the medical system further comprises a third expandable portion capable of being in fluid communication with the first and second expandable portions, and further comprising positioning the third expandable portion spaced from the vertebras.

33. The method of claim 32, wherein the third expandable portion is positioned posterior of the vertebras.

34. The method of claim 21, farther comprising expanding a test balloon between the vertebras.

35. The method of claim 34, further comprising introducing a fluoroscopically visible agent into the balloon.

36. The method of claim 21, further comprising introducing a bone morphogenic material into the first expandable portion.

37. The method of claim 21, further comprising securing the second expandable portion to a spinal process.

38. The method of claim 21, further comprising securing the second expandable portion in-line between two spinal processes.

39. A method, comprising:
removing at least a portion of a disc in a disc space between two vertebras;
using a posterior approach to position a first expandable portion of a medical device system in the disc space between the two vertebras; and
using a posterior approach to position a second expandable portion of the medical device system posterior to the disc space.

40. The method of claim 39, wherein the disc is removed bilaterally.

41. The method of claim 39, further comprising expanding a test balloon in the disc space after removing a portion of the disc and prior to positioning the first expandable portion of the medical device system.

42. The method of claim 41, further comprising introducing a fluoroscopically visible agent into the balloon.

43. The method of claim 39, further comprising securing at least one screw to at least one of the vertebras.

44. The method of claim 39, further comprising removing at least a portion of a facet joint of at least one of the two vertebras.

45. The method of claim 39, further comprising constraining a portion of the medical device system from extending.

46. The method of claim 39, further comprising securing the second expandable portion to a screw secured to a vertebra.

47. The method of claim 39, further comprising introducing a fluid into the first expandable portion of the second expandable portion.

48. The method of claim 47, further comprising adjusting the amount of the fluid in the medical device system.

49. The method of claim 48, wherein the pressure is adjusted from a posterior approach through a valve in fluid communication with the first and second expandable portions.

50. The method of claim 39, further comprising introducing bone morphogenic material into the first expandable portion.

51. The method of claim 39, further comprising using a posterior approach to position a third expandable portion of the medical device system posterior to the disc space, the third expandable portion capable of being in fluid communication with the first and second expandable portions.

52. The method of claim 39, further comprising using a posterior approach to position a third expandable portion of the medical device system in the disc space between the vertebras, the third expandable portion capable of being in fluid communication with the first and second expandable portions.

53. The method of claim 39, further comprising using a posterior approach to position a third expandable portion of the medical device system in the disc space between the vertebras, and using a posterior approach to position a fourth expandable portion of the medical device system, the third and fourth expandable portions capable of being in fluid communication with each other.

54. The method of claim 39, wherein the first expandable portion contacts an annulus between the two vertebras.
